(19) United States
(12) Patent Application Publication
(10) Pub. No.: US 2016/0340741 A1
(43) Pub. Date: Nov. 24, 2016

(54) MUTATIONS DEFINE CLINICAL SUBGROUPS OF GLIOMAS

(71) Applicant: DUKE UNIVERSITY, Durham, NC (US)

(72) Inventors: Hai Yan, Durham, NC (US); Darell Bigner, Durham, NC (US); Patrick Killela, Durham, NC (US); Zachary Reitman, Durham, NC (US)

(73) Assignee: Duke University, Durham, NC (US)

(21) Appl. No.: 15/115,044

(22) PCT Filed: Jan. 26, 2015
(86) PCT No.: PCT/US15/12888

§ 371 Date: Jul. 28, 2016

Related U.S. Application Data
(60) Provisional application No. 61/932,291, filed on Jan. 28, 2014.

(51) Int. Cl. C12Q 1/68

(52) U.S. Cl. CPC C12Q 1/6886; C12Q 2600/112; C12Q 2600/118; C12Q 2600/156

(57) ABSTRACT

Genetic signatures capable of distinguishing among several types of gliomas provide clinically relevant information that can serve as an adjunct to histopathological diagnosis. For example, mutations in the TERT promoter occurred in 74.2% of glioblastomas (GBM), but occurred in a minority of Grade II-III astrocytomas (18.2%). In contrast, IDH1/2 mutations were observed in 78.4% of Grade II-III astrocytomas, but were uncommon in primary GBM. The genetic signatures permit the stratification of the glioma patients into distinct cohorts.

This invention was made with government support under grant no. 1R01-CA1403160 awarded by the National Cancer Institute. The government has certain rights to this invention.

### TECHNICAL FIELD OF THE INVENTION

This invention is related to the area of oncology. In particular, it relates to cancer characterization and management.

### BACKGROUND OF THE INVENTION

Gliomas are the most common primary malignant tumor of the central nervous system and account for 24% of brain tumors [1]. Tumor grades range from Grade I to Grade IV and are based on histopathological and clinical criteria established by the World Health Organization (WHO) [1, 2]. Grade I tumors are relatively benign and are circumscribed tumors that display a favorable prognosis with 94% of patients surviving at 5 years and 91% at 10 years [1]. Grade II gliomas are diffusely infiltrative and can be divided into astrocytomas and oligodendrogliomas. These tumors have the inherent ability to progress to higher grade gliomas. In addition to Grade II and III astrocytomas and oligodendrogliomas, another subtype of glioma presents with a histological appearance of both oligodendrogliomas and astrocytomas. These “mixed histology” tumors, or oligoastrocytomas also have the ability to progress from Grade II to Grade III tumors. The Grade III astrocytomas have the ability to further progress into secondary Grade IV glioblastomas (GBM), which exhibit a poorer prognosis than the grade III astrocytomas. As first described by Scherer in 1940 [3] secondary GBM arises as a progression from Grade II and Grade III tumors, whereas primary GBM arises de novo and has a dismal median OS of 15 months [1]. The progression between grades along with the potential for mixed histology presents neuropathologists with diagnostic challenges that often rely on subjective measures. Consequently, diagnoses among different pathologists and institutions have weak correlations that may result in variable treatment and management of each tumor grade [2, 4]. The subjective nature of these analyses stresses the importance of an accurate, unbiased, and objective means of diagnosis. This is crucial for stratification of patients with biologically similar tumors in clinical trials, and could aid in the selection of targeted therapeutic regimens. The discovery of biomarkers that objectively identify each tumor's unique molecular signature is a necessary next-step in managing patient outcomes more effectively. Genetic signatures performed on pathologically relevant tissues will be a potentially useful supplement to clinicians in refining and clarifying patient stratification.

[04] Characterization of the genetic landscape of gliomas has been at the forefront of cancer research in order to better aid prognostication and classification of clinical outcomes [5, 6]. High-throughput screens have paid particular attention to understanding the genomic variability between each subgroup of glioma. The Cancer Genome Atlas and other groups, including ours, have begun to identify the molecular subgroups of these tumors and delineate which tumor types harbor which mutations [5-12]. For example, IDH1/2 mutations that occur frequently in secondary GBMs (>50%) are infrequent in primary GBMs (<5%) [8, 12].

Recent findings have established frequent mutations in the promoter region of telomerase reverse transcriptase (TERT) in a multitude of cancers, including melanomas, liposarcomas, bladder cancer, urinary tract cancers, and gliomas [13-19]. TERT is a subunit of the telomerase enzyme that, when expressed, allows cells to avoid senescence. This is especially noted as TERT is mutated in high frequencies in cells with low rates of self-renewal, such as melanocytes, urothelial cells, and glial cells [14-16, 20, 21]. Of interest to glioma genomics, TERT promoter mutations occur in 70-80% of primary GBMs and >70% of oligodendrogliomas, but occur less frequently in both lower grade astrocytomas and most oligoastrocytomas [16, 17, 22].

There is a continuing need in the art to develop better means for analyzing and characterizing cancers so that the patients can obtain optimal quality of life and outcomes.

### SUMMARY OF THE INVENTION

According to one aspect of the invention a method is provided for characterizing brain tumors of patients. A sample from a brain tumor patient is tested for TERT promoter, IDH1, and IDH2 to determine mutation status at mutational hot spots C228 and C250 of the TERT promoter, at residue R132 of IDH1 or the codon encoding said residue, and at residue R172 of IDH2. A patient sample is assigned to a group that has the same mutational status as the patient sample.

[08] Another aspect of the invention is a device for characterizing brain tumors. It comprises one or more solid supports which comprise hybridization probes which hybridize to polynucleotide fragments comprising IDH1 codon 132, IDH2 codon 172, and TERT promoter mutation hot spots C228 and C250.

[09] Another aspect of the invention is a kit for detecting IDH1, IDH2, and TERT promoter mutations. The kit comprises nucleic acid primers and/or nucleic acid probes which specifically amplify or hybridize to IDH1, IDH2, and TERT promoter mutation hot spots IDH1 R132, IDH2 R172, TERT promoter C228, and TERT promoter C250.

These and other embodiments which will be apparent to those of skill in the art upon reading the specification provide the art with

### BRIEF DESCRIPTION OF THE DRAWINGS

FIGS. 1A-1D: Distribution of TERT promoter and IDH1/2 mutations in a panel of 473 adult gliomas. Mutational analysis of 473 adult gliomas for TERT promoter and IDH1/2 mutations. Data are from 240 Grade IV GBM (FIG. 1A), 88 Grade II-III astrocytomas (FIG. 1B), 58 Grade II-III oligoastrocytomas (FIG. 1C), and, 87 Grade II-III oligodendrogliomas (FIG. 1D). Mutation status is indicated by color shading, with gray coloring indicating wild type sequence, red (top row) indicating mutations in the TERT promoter, and green (bottom row) indicating mutations in IDH1/2.

FIG. 2A-2C: Overall Survival stratified by TERT promoter and IDH1/2 mutational status and histology within each tumor grade. Overall survival was represented by Kaplan Meier plots for individual WHO tumor grade: FIG. 2A) Grade II (n=103), FIG. 2B) Grade III (n=121), FIG. 2C) Grade IV (n=218). Only subgroups with at least 10 patients were included in the analyses. Tumors were represented by mutations status on the left (TERT promoter status/IDH1/2 status) and histology on the right (A represents Astrocytomas, O represents Oligodendrogliomas, and OA represents Oligoastrocytomas).

FIG. 3A-3B: Overall Survival stratified by TERT promoter and IDH1/2 mutational status and histology among Grade III and IV patients. Overall survival was represented by Kaplan Meier plots stratified by FIG. 3A) histology (A represents Astrocytomas, O represents Oligodendrogliomas, OA represents Oligoastrocytomas, and GBM represents Glioblastoma) and FIG. 3B) TERT promoter/IDH1/2 mutation status for all Grade III and Grade IV gliomas analyzed in this study.

FIG. 4 (Table 1) Clinical characteristics of cohort

FIG. 5 (Table 2) Age at diagnosis in gliomas as determined by TERT promoter genotype

FIG. 6. (Table 3) Summary of OS stratified by TERT promoter and IDH1/IDH2 mutational status by grade

FIG. 7 (Table 4) Cox Model predicting median overall survival in GBMs

FIG. 8 (Tables 5A) Summary of OS stratified by histology in grades III and IV.

FIG. 9 (Table 5B) Summary of OS stratified by TERT promoter and IDH1/IDH2 mutational status in grades III and IV.

### DETAILED DESCRIPTION OF THE INVENTION

The inventors have assessed the characteristic variation between IDH1/2 and TERT promoter mutations among several glioma subtypes. This is useful inter alia to help refine the diagnosis of gliomas. One assay that can be used to determine a glioma subtype is based upon three polymerase chain reactions (PCR). It can provide pathologists with a manageable and reliable diagnostic tool in the form of a simple, yet robust genetic signature unique to each tumor type. Other assays may be used.

Determination of mutation status at the TERT promoter hotspots and at the IDH1/IDH2 hotspot for mutations can be performed by any technique known in the art. The relevant portions of the genome from the patient sample can optionally be amplified. The amplified product can be determined by sequencing, by hybridization to a mutation or wild type specific probe, by mutation specific amplification, by single base extension, etc. In the case of the IDH1/IDH2 mutations, these can also be determined by using immunological techniques, such as immunohistochemistry. The IDH1/IDH2 mutation hotspots are each in exon 4, which can be readily amplified and assayed. Typically a single mutation in either of the TERT promoter hotspots and in either of IDH1 or IDH2 will be sufficient to categorize the marker as mutant.

A solid support (or a collection of solid supports, such as beads) can be used to capture relevant polynucleotides for testing for mutational status. The solid support may comprise hybridization probes to capture by hybridization polynucleotides from the patient sample that can be subsequently analyzed or analyzed by virtue of their capture by a specific probe. Typically the solid support or collection of solid supports will comprise probes for less than 25 different genes, for less than 20 different genes, for less than 15 different genes, for less than 10 different genes, or for less than 5 different genes. Multiple forms of a gene may be present, however, to capture different forms of the queried gene.

The solid supports may be in any form, including arrays, plates, beads, dipstick, etc. If beads are used, they may optionally be magnetic. Any such supports as are known in the art may be utilized for their known purposes. Multiple supports may be attached to each other or contained within a single vessel, if desired.

If hybridization is the means of identification, it may be useful to employ detectable probes, such as labeled probes or secondary reagents that will bind to the probe to render the probe detectable. Suitable labels which may be use include any known in the art including those which are radiolabeled, chromogenically labeled, fluorescently labeled, etc.

Probes and primers may be designed to hybridize and be complementary to the precise nucleotide where a mutation resides or adjacent to such nucleotide. If adjacent, a second step can be used to determine the identity of the nucleotide of interest. An example of a second step is a single base extension reaction. The invention is not however, limited to such examples.

Kits are used to package two or more components in a single commercial unit, typically a box or carton. There may be multiple containers within the single commercial unit. Information in the form of printed matter, an electronic information storage medium such as a disk or drive, or a reference to a cloud based document may be used. Devices and reagents may be combined in a kit or only devices or only reagents. The kit may be used to carry out a single type of process or a series of processes that are typically interrelated. Kits may contain reagents necessary for carrying out particular reactions such as polymerase chain reactions, sequencing reactions, rolling circle amplification, single base extension reactions, etc. Sequencing reactions may be, for example, next generation sequencing reactions or Sanger sequencing.

Groups to which patients may be assigned may be those which have a similar prognosis. They may be those that will be in a single clinical trial group or arm. They may be those that are good candidates for a particular therapeutic agent or regimen. Prognosis may be based on overall survival, recurrence rates, or disease free survival periods, as examples.

Our analysis of this tumor cohort expands upon previous reports identifying frequent

TERT promoter mutations in gliomas [16-18, 22, 23], examines the association between TERT promoter and IDH1/2 mutations in glioma, and assesses their joint influence on OS. Utilizing a combined analysis of IDH1/2 and TERT promoter mutations in adult glioma, we have derived a greatly expedited and simplified genetic signature of three common glioma subtypes, namely Grade II-III astrocytomas, oligodendrogliomas, and GBMs. Additionally, we show that oligoastrocytomas can be further classified.

Among patients with GBMs, we showed that the largest fraction of GBMs present with TERT promoter mutations. IDH1/2 mutations are infrequent in these tumors and cluster within secondary GBMs. Three distinct subgroups were defined by the presence or absence of TERT promoter and IDH1/2 mutations. Where patients harboring tumors with TERT promoter mutations alone had the poorest OS (median 11.3 months), patients with tumors bearing no mutations in either TERT or IDH1/2 had a slightly better survival (median 16.6 months), and GBMs with IDH1/2 mutation alone resulted in the best survival (median 42.3 months). Furthermore, these associations remained after adjustment for factors such as age. TERT promoter mutations predicted poorer OS outcome in a multivariate model even in GBMs without IDH1/2 mutations. This finding is in contrast with previous studies that did not report a significant difference in OS between TERT promoter-mutated and TERT promoter-wildtype non IDH mutated GBMs [23]. This finding will be of particular interest to clinicians as it may provide a tool to stratify non IDH1/2 mutant GBMs and suggests that combined IDH1/2 and TERT promoter genotyping will be useful for patient management. Because of variable treatment among these histological brain tumor groups, further analyses must include large cohorts of standardized treatment arms and measurements of other genetic features such as MGMT status, EGFR wildtype amplification, and the presence of EGFRvIII to confirm the validity of our findings. At a minimum, our current findings warrant further investigation and confirmation by other investigators. Also, genetic alterations of the TERT promoter may be particularly relevant given the development of therapeutics targeted against telomerase. Telomerase inhibitors have shown promise for treating GBM in preclinical models and are currently under investigation in clinical trials for several types of cancer [24-27].

Conversely, IDH1/2 mutations in Grade II-III astrocytomas are frequent while TERT promoter mutations are uncommon. Grade II-III oligodendrogliomas have frequent co-occurring mutations in the TERT promoter and IDH1/2. We provide evidence that over 86% of oligoastrocytomas in this cohort contain genetic signatures representative of either astrocytoma (IDH1/2 mutations alone) or oligodendroglioma (TERT promoter/IDH1/2), signatures that we show are associated with OS. Reproducibility of oligoastrocytoma diagnosis by histology alone displays variable diagnoses between neuropathologists within and among different institutions [2, 4, 28]. The presence of the TERT promoter and IDH1/2 mutational status may be particularly useful to refine the classification of “mixed” oligoastrocytomas.

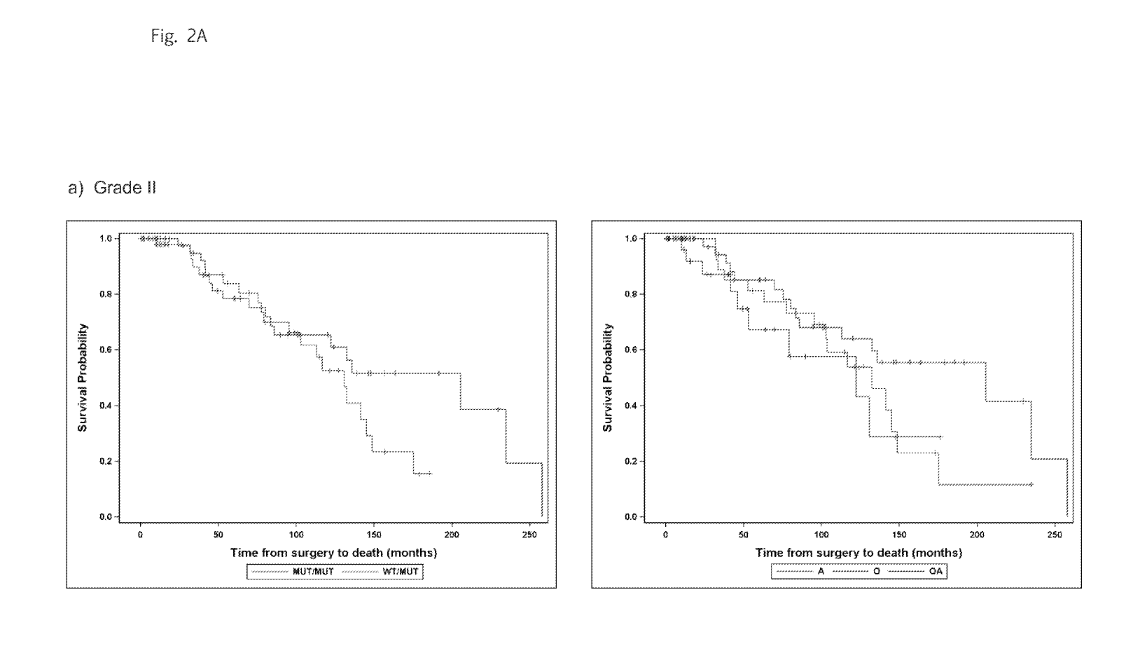

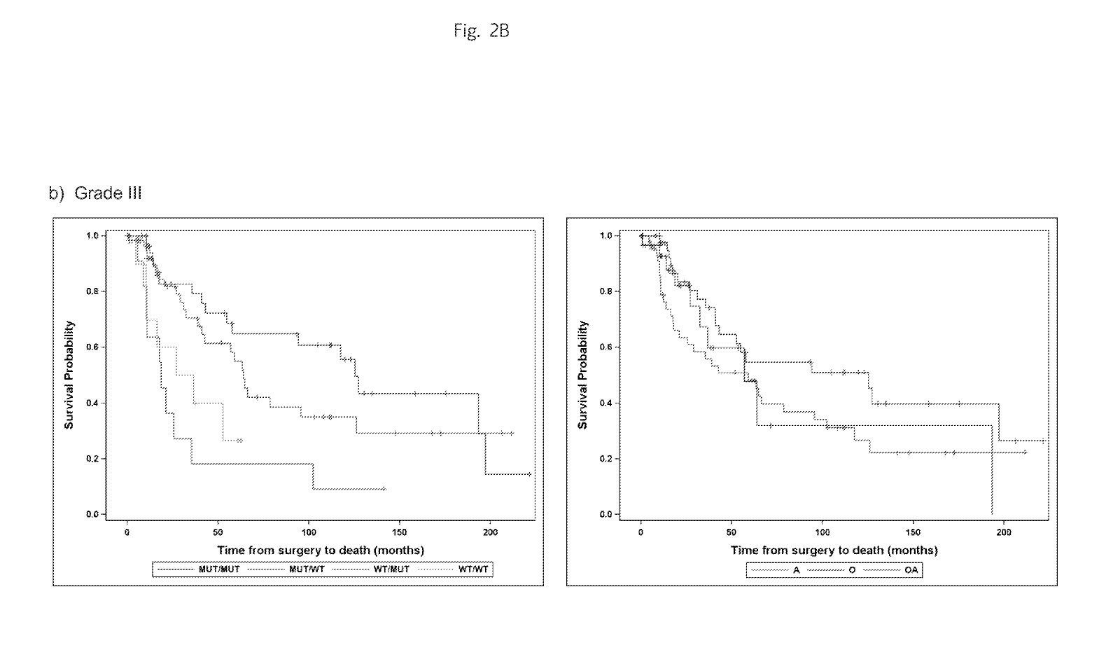

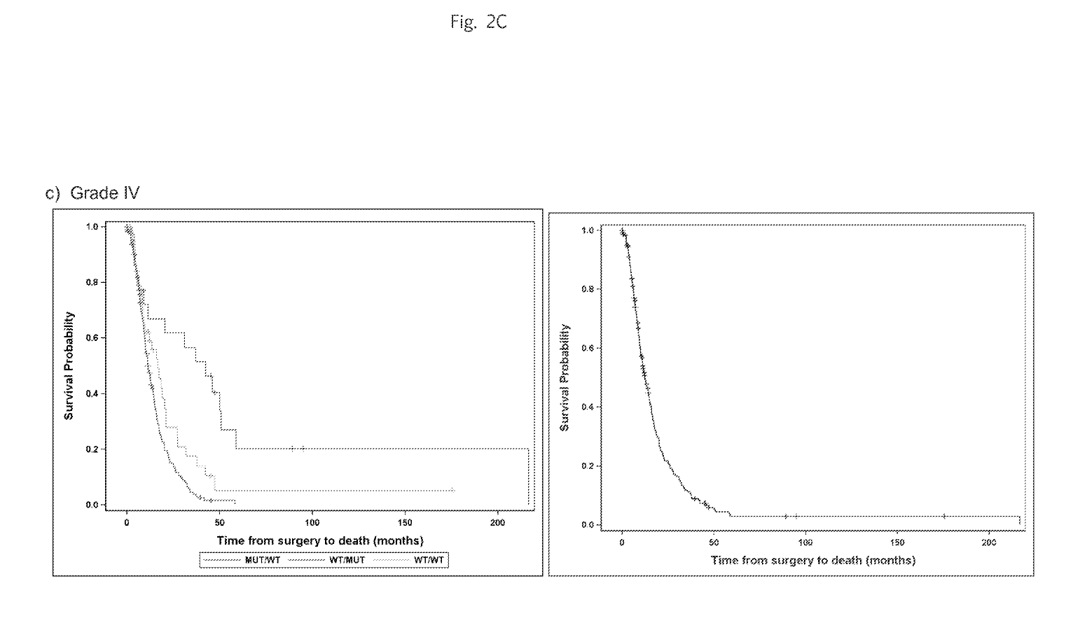

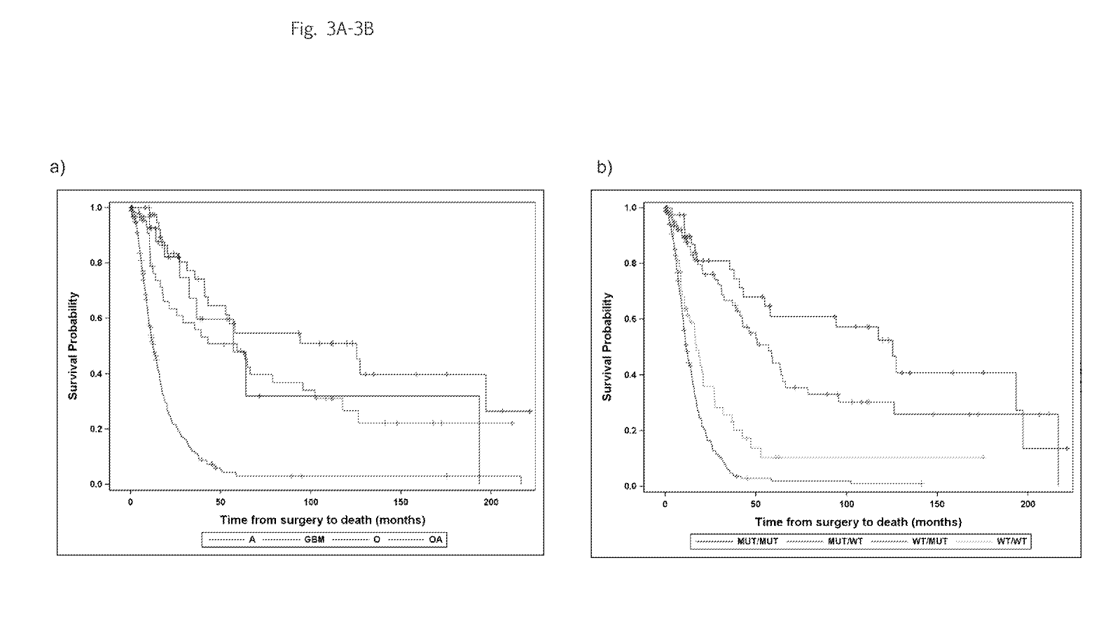

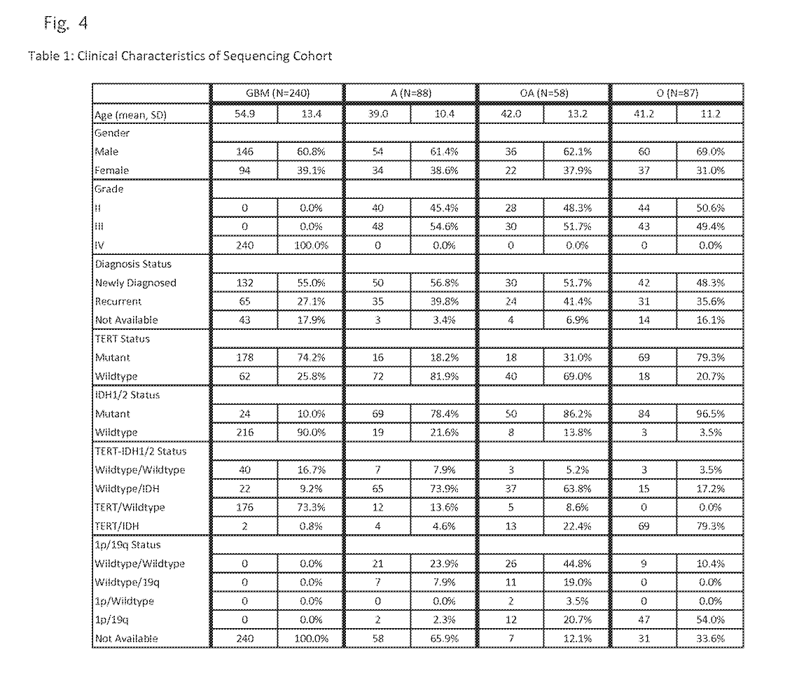

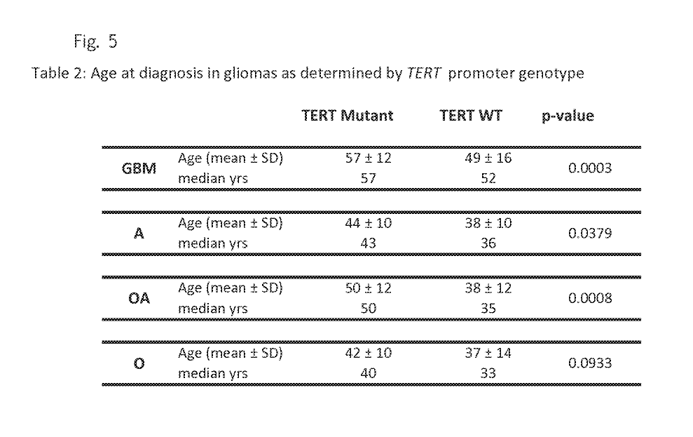

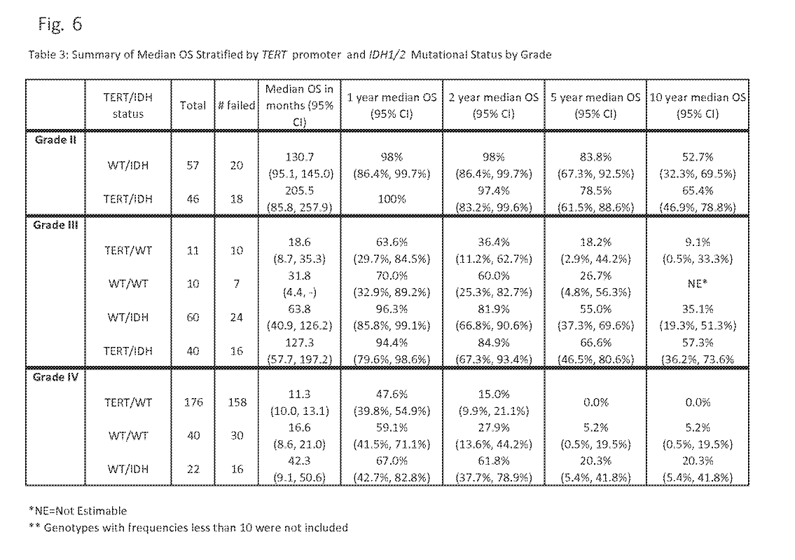

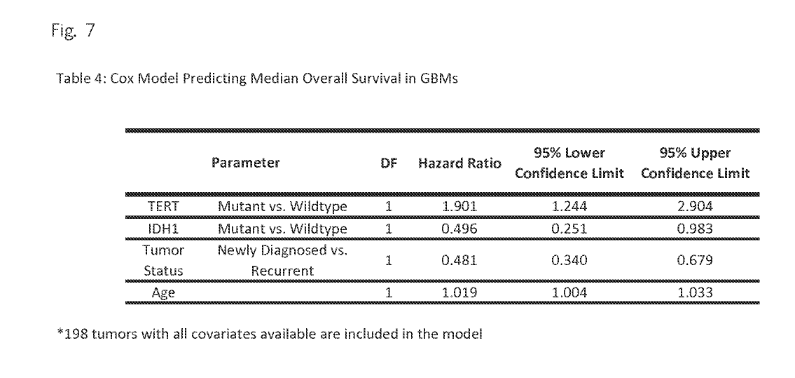

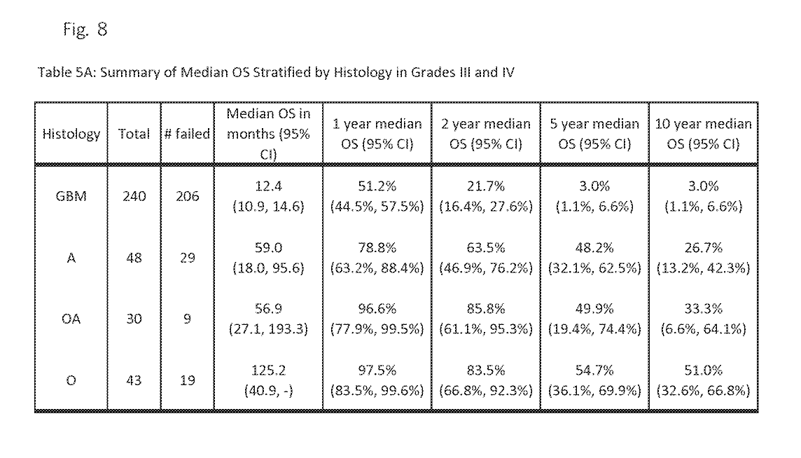

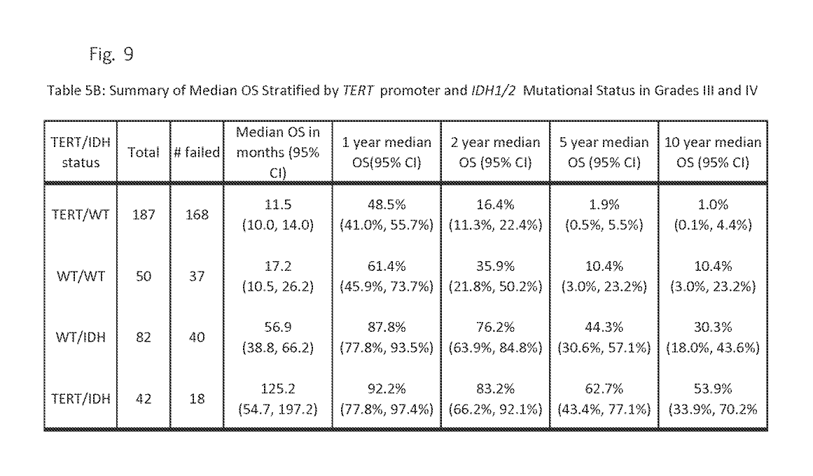

In addition to demonstrating the robust nature of these mutational patterns, we have further established that these genetic signatures are reliable when compared to the OS of patients derived from conventional histopathological diagnosis. As shown in FIGS. 2 and 3, mutations in the TERT promoter and IDH1/2 effectively stratify patients into reproducible subgroups based on survival. This phenomenon was independent of grade among high grade astrocytomas as Grade III and Grade IV tumors mimicked this relationship when analyzed independently (FIG. 2). Furthermore, the strength of these genetic signatures and their association with OS is illustrated by a slightly higher R² (0.3132 vs. 0.2704) than by histology alone.

Two clinical subgroups exist among Grade II tumors in the current cohort, as the power of the survival analysis was limited due to the smaller number of low grade gliomas. The Grade II tumors exhibited genetic signatures with mutations in IDH1/2 alone, and tumors with mutations in the TERT promoter and IDH1/2. Both subgroups had a more favorable prognosis, with a median OS of 130.7 months in tumors with IDH1/2 mutations alone, and median OS of 205.5 months among patients whose tumors harbored TERT promoter and IDH1/2 mutations. No Grade II tumors exhibited TERT promoter mutations alone.

Within Grade III-IV gliomas, those patients with the TERT promoter mutations alone had the poorest prognosis (median 11.5 months), while tumors bearing the events typically representative of astrocytomas (IDH1/2 mutation) had a more favorable prognosis (median 56.9 months). Tumors harboring mutations typically seen in oligodendroglioma (both TERT promoter and IDH1/2 mutation) had a more favorable prognosis (median 125.2 months). Tumors that did not harbor mutations in either the TERT promoter or IDH1/2 comprised a unique clinical group with a short OS (median OS 17.2 months) that was distinct from TERT promoter mutated gliomas (median OS 11.5 months) (Table 5B). As these gliomas, wild-type for both TERT promoter and IDH1/2 mutations, represented a clinically distinct unit (FIGS. 2B and 2C) further investigation is required to delineate critical driver mutations in this subset of gliomas.

It is of interest to note that within each tumor type, a minority of tumors bore the genetic signature typically associated with other histological subtypes. In particular, 13.6% (12/88) of Grade II-III astrocytomas bore TERT promoter mutations alone and occasional Grade II-III astrocytomas harbored both TERT promoter and IDH1/2 mutations (4/88, 4.6%). This suggests that at least genetically, these tumors may be more similar to GBM and oligodendroglioma, respectively. Oligodendrogliomas were almost exclusively TERT promoter and IDH1/2 mutated (79.3%, 69/87), but a fraction, 17.2% (15/87) harbored mutations in IDH1/2 alone. In our cohort, no oligodendroglioma cases harbored TERT promoter mutations alone. A minor fraction of GBMs (0.8%, 2/240) contained mutations in both the TERT promoter and IDH1/2 suggesting they were treated oligodendrogliomas that were diagnosed as small cell GBMs.

Loss of chromosomal arms 1p and 19q is a well-known genetic event associated with oligodendrogliomas that many neuropathologists use as a reliable test for diagnosing oligodendroglioma, a tumor generally associated with favorable prognosis and response to chemotherapy [29-32]. As a secondary analysis, the 69 oligodendrogliomas with 1p/19q status available were analyzed for an association with TERT promoter/IDH1/2 mutational status. All 44 oligodendrogliomas with TERT promoter and IDH1/2 mutations also had the 1p/19q allelic deletions and all but 3 of the 47 tumors with 1p/19q allelic losses also contained both TERT promoter and IDH1/2 mutations, indicating that IDH1/2 and TERT promoter mutational analysis may be a comparable prognostic markers to 1p and 19q in oligodendrogliomas (Fisher exact p<0.0001).

This study supports genotyping of TERT promoter and IDH1/2 in gliomas as a rapid, cost-effective test requiring little tumor DNA that could help inform clinicians as to the predicted OS of these tumors that may differ from their predicted outcomes based on conventional histology alone. The TERT promoter mutations analyzed in this study lay only 22 base pairs apart, allowing for PCR amplification in a single amplicon.

Additionally, the most frequent mutations in IDH1 and IDH2 occur in hotspot residues located at resides R132 and R172, respectively. Combined together, these three PCR amplicons allow for expedient turnaround, objective interpretation, and vast economic advantages to glioma patients.

The TERT promoter/IDH1/2 mutational profiles of each tumor type can be used in several aspects of the clinical process including stratification of patients, examination of therapeutic response, and selection of treatment, among others. Given the background genes previously discovered in glioma, we hypothesize TERT promoter and IDH1/2 mutations as the major driver genes that are consistently found in low-grade and high-grade adult gliomas. These gene mutation assays will support and expedite the diagnosis of brain tumors while supplementing histopathological evaluation. Measurement of these biomarkers could further increase the fidelity of glioma diagnosis in a rapid and cost-effective manner. Furthermore, the simplicity and affordability of these tests underscore their importance as a tool to aid neuropathologists in glioma diagnosis. Notably, these signatures can be applied to cases that present atypical morphologic features in standard histopathological analysis. Taken together these findings simplify the genetic classification of glioma. The ability of these genetic signatures to stratify patients will refine and clarify the diagnostic accuracy of pathologists by supplementing standard histopathological criteria with genetic mutational analysis.

The above disclosure generally describes the present invention. All references disclosed herein are expressly incorporated by reference. A more complete understanding can be obtained by reference to the following specific examples which are provided herein for purposes of illustration only, and are not intended to limit the scope of the invention.

### EXAMPLE 1

### Methods

### Sample Collection, Processing, and Sequencing

Adult glioma (18> years old) and corresponding clinical information were obtained with consent and Institutional Review Board approval from the Preston Robert Tisch Brain Tumor Center BioRepository at Duke University in accordance with the Health Insurance Portability and Accountability Act. Newly diagnosed versus recurrent glioma status and vital status were determined by clinical chart review. Fresh frozen tissue sections (first and last sections from the block, stained with hematoxyline and eosin) were reviewed by a board-certified neuropathologist (REM) to confirm original clinical histopathologic diagnosis and to ensure intervening studied sections contain >80% tumor cells. DNA was extracted from 240 Grade IV GBMs, 88 Grade II and Grade III astrocytomas, 58 Grade II and Grade III oligoastrocytomas, and 87 Grade II and Grade III oligodendrogliomas. Of the 473 tumors, 160 gliomas had been analyzed in our previous studies of the TERT promoter [16]. Isolated DNAs were PCR amplified for the TERT promoter, exon 4 of IDH1, and analyzed via Sanger sequencing for 473 tumors as described previously [12, 16, 33]. Additionally, on those cases that did not harbor mutations in IDH1 we amplified exon 4 of IDH2 and analyzed them via Sanger sequencing. 1p and 19q copy number was evaluated by microsatellite marker analysis and via 1p and 19q FISH testing in a certified clinical laboratory as described previously [12, 29, 34].

### Statistical Methods

Clinical and demographic characteristics at the time of diagnosis were summarized for all patients and stratified by histologic tumor type. Means and standard deviations were used to describe interval variables, whereas frequency distributions were used to describe categorical variables. Unpaired t-tests were used to compare the mean age of patients with and without TERT promoter mutations. The Kaplan-Meier estimator was used to describe OS. OS was defined from time of surgery to death or last follow-up. Multivariable Cox models were used to assess the effect of TERT promoter and IDH1/2 mutations on OS adjusting for baseline tumor characteristics. The generalized R2 statistic was used to assess the strength of association between covariates. Associations between categorical variables were analyzed using Fisher exact tests.

### EXAMPLE 2

### TERT Promoter Mutations are Frequent in Primary GBMs and Pligodendrogliomas but Uncommon in Lower Grade Astrocytoma

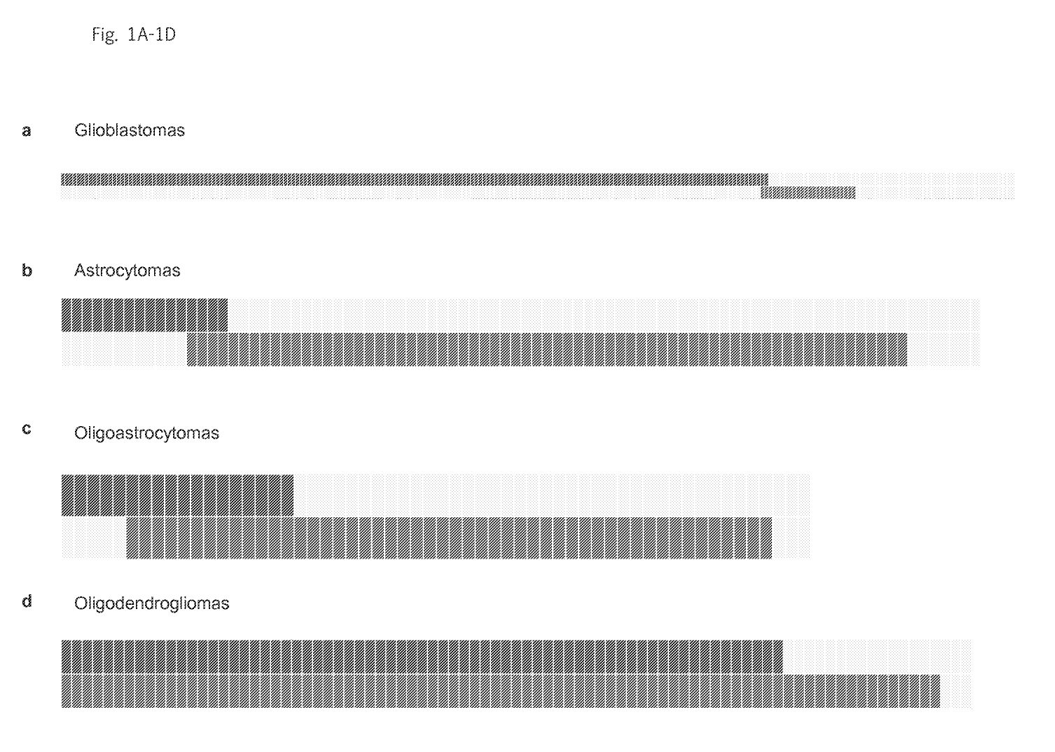

To assess the prevalence and prognostic impact of TERT promoter mutations we sequenced the proximal TERT promoter hotspot mutations (C228T and C250T) in 473 adult gliomas. We identified TERT promoter mutations in 281 (59.4%) tumors (FIG. 1). In agreement with previous studies [16, 18, 23], we identified TERT promoter mutations in 74.2% of grade IV GBMs (178/240). TERT promoter mutations were also common in oligodendrogliomas (79.3%); however, TERT promoter mutations were less frequently identified in Grade II-III astrocytomas (18.2%, 16/88). Furthermore, we observed a moderate frequency of TERT promoter mutations in oligoastrocytomas (31.0%, 18/58). As expected, GBMs were diagnosed in older patients when compared to other histologic subtypes studied here (Table 1). Within each tumor type, TERT promoter mutations were associated with an older age at diagnosis (Table 2).

### EXAMPLE 3

### Co-occurring Mutations in TERT Promoter and IDH1/2

IDH1/2 mutations are a well-established molecular feature of gliomas [12]. To define the co-occurrence of IDH1/2 mutations and the presence of TERT promoter mutations, we determined the status of IDH1 and IDH2 mutations in the same cohort of 473 gliomas and identified mutations in 47.9% (227/473) of tumors (FIG. 1 and Table 1). IDH1/2 mutations were much less prevalent among GBMs (10%), and much more common in Grade II-III astrocytomas (78.4%), oligoastrocytomas (86.2%) and oligodendrogliomas (96.5%). TERT mutations occurred in the absence of IDH1/2 mutations in GBMs (73.3%, 176/240). However, in oligodendrogliomas, the TERT promoter mutation always occurred in the setting of the IDH1/2 mutation, which is frequent in both oligodendrogliomas and astrocytomas (FIG. 1) [12]. The cross-tabulation of TERT promoter and IDH1/2 mutations aligned with three of the four histologic subtypes. GBMs were characterized as primarily TERT promoter mutant/IDH wildtype (73.3%), Grade II-III astrocytomas were predominantly TERT promoter wildtype/IDH mutant (73.9%), and the majority of oligodendrogliomas mainly harbored mutations in both the TERT promoter and IDH1/2 (79.3%). A majority of oligoastrocytomas (63.8%) exhibited the IDH mutation in the absence of TERT promoter mutations, much like Grade II-III astrocytomas; however, a fraction (22.4%) of oligoastrocytomas presented with both TERT promoter and IDH1/2 mutations, similar to oligodendrogliomas (FIG. 1).

### EXAMPLE 4

TERT Promoter and IDH1/2 Mutations have Distinct Tumor Distributions and are Associated with OS

We next sought to determine whether the combination of TERT promoter and IDH1/2 mutations are associated with OS. Clinical information (survival, age at diagnosis, and histopathological diagnosis) was available for our cohort of 473 adult gliomas in both treated and untreated patients (Table 1). As grade is a well-known prognostic factor in glioma patients, we first investigated whether distinct tumor subgroups could be distinguished using only TERT promoter and IDH1/2 mutation status within each grade (FIG. 2, Table 3). Among the 112 Grade II gliomas, 103 were characterized by either mutations in both TERT and IDH or IDH alone. The median OS of those tumors harboring mutations in both TERT promoter and IDH1/2, the predominant genetic signature in oligodendrogliomas, was longer than those tumors with an IDH1/2 mutation only, typically seen in Grade II-III astrocytomas (206 months vs. 131 months), but this difference was not statistically significant (log-rank p=0.1754) (FIG. 2A). When stratified by histologic diagnosis, oligodendrogliomas had the best median OS among Grade II astrocytomas, oligodendrogliomas, and oligoastrocytomas, as expected (median OS 205 months).

Among the 121 Grade III tumors, 60 (50%) had IDH1/2 mutations alone and 40 (34%) had mutations in both the TERT promoter and IDH1/2. Those with mutations in both the TERT promoter and IDH1/2 had the largest median OS (127 months), followed by those with an IDH1/2 mutation only (median OS 64 months), and those with neither mutation (median OS 32 months). Tumors with mutations in the TERT promoter alone, which was the predominant signature present in primary GBMs had the poorest OS (median OS 19 months). Four distinct subgroups of Grade III gliomas were identified when stratified by the combination of the TERT promoter and IDH1/2 mutation status (log-rank p=0.0008) (FIG. 2B). Oligodendrogliomas again had the best median OS when Grade III tumors were stratified by histologic subtypes (median OS 125 months), but OS did not significantly differ among the three histologic subtypes, which were astrocytomas, oligodendrogliomas, and oligoastrocytomas (log-rank p=0.1626).

A majority of the GBMs were characterized by mutations in the TERT promoter alone (73%), and this genetic signature also had the worst prognosis (median OS 11.3 months) (FIG. 2, Table 3). Those without mutation in either marker had only a slightly better outcome (median OS 17 months), while those with an IDH1/2 mutation alone, the signature characteristic of Grade II-III astrocytomas and Grade IV secondary GBMs, had the best outcome among the Grade IV tumors (median OS 42 months). Within the primary and secondary GBMs, using the TERT promoter and IDH1/2 alone, we were able to distinguish three significantly different subgroups (log-rank p<0.0001), and these associations remained when adjusting for the factors of age and diagnosis (Table 4). The TERT promoter mutation is associated with poorer OS in GBMs, and as shown in the multivariable model, this association was also evident among tumors without an IDH1/2 mutation (HR: 1.9, 95% CI: 1.2-2.9).

Given that both Grade III and Grade IV gliomas were successfully stratified into distinct subgroups based on TERT promoter and IDH1/2 mutational status, and that each signature was associated with a similar median OS within grade, the effect of histology and genetic signature on OS was also examined across the Grade III and IV gliomas together (FIG. 3, Table 5A). When Grade III and IV gliomas were examined based on histology, GBMs predictably had by far the worst prognosis, and oligodendrogliomas experienced the best survival outcome; however, OS among the Grade III astrocytomas and oligoastrocytomas was similar and difficult to distinguish (FIG. 3A and Table 5A). Nevertheless, when genetic signatures were applied to the same cohort of tumors, four distinct clinical subgroups emerged (FIG. 3B). As observed, within Grade III and IV gliomas separately, tumors with mutations in both TERT and IDH1/2 had the best median OS (oligodendroglioma signature), followed by those with an IDH1/2 mutation only (Grade II-III astrocytoma and secondary GBM signature). Both tumors without mutation in either marker and those tumors with a TERT promoter mutation alone had a poorer prognosis, with the latter signature having the worst median OS. The strength of the association between OS and TERT/IDH1/2 mutational status is similar to that of OS and histology (Generalized R2: 0.3132 and 0.2704, respectively).

### REFERENCES

The disclosure of each reference cited is expressly incorporated herein.

1. Dolecek T A, Propp J M, Stroup N E and Kruchko C. CBTRUS statistical report: primary brain and central nervous system tumors diagnosed in the United States in 2005-2009. Neuro-oncology. 2012; 14 Suppl 5:v1-49.

2. Louis D N, Ohgaki H, Wiestler O D, Cavenee W K, Burger P C, Jouvet A, Scheithauer B W and Kleihues P. The 2007 WHO classification of tumours of the central nervous system. Acta neuropathologica. 2007; 114(2):97-109.

3. Scherer H J. Cerebral Astrocytomas and Their Derivatives. The American Journal of Cancer. 1940; 40(2):159-198.

4. Wen P Y and Kesari S. Malignant gliomas in adults. The New England journal of medicine. 2008; 359(5):492-507.

5. Cancer Genome Atlas Research N. Comprehensive genomic characterization defines human glioblastoma genes and core pathways. Nature. 2008; 455(7216):1061-1068.

6. Parsons D W, Jones S, Zhang X, Lin J C, Leary R J, Angenendt P, Mankoo P, Carter H, Siu I M, Gallia G L, Olivi A, McLendon R, Rasheed B A, Keir S, Nikolskaya T, Nikolsky Y, et al. An integrated genomic analysis of human glioblastoma multiforme. Science. 2008; 321(5897):1807-1812.

7. Brennan C W, Verhaak R G, McKenna A, Campos B, Noushmehr H, Salama S R, Zheng S, Chakravarty D, Sanborn J Z, Berman S H, Beroukhim R, Bernard B, Wu C J, Genovese G, Shmulevich I, Barnholtz-Sloan J, et al. The Somatic Genomic Landscape of Glioblastoma. Cell. 2013; 155(2):462-477.

8. Jiao Y, Killela P J, Reitman Z J, Rasheed A B, Heaphy C M, de Wilde R F, Rodriguez F J, Rosemberg S, Oba-Shinjo S M, Nagahashi Marie S K, Bettegowda C, Agrawal N, Lipp E, Pirozzi C, Lopez G, He Y, et al. Frequent ATRX, CIC, FUBP1 and IDH1 mutations refine the classification of malignant gliomas. Oncotarget. 2012; 3(7):709-722.

9. Johnson B E, Mazor T, Hong C, Barnes M, Aihara K, McLean CY, Fouse S D, Yamamoto S, Ueda H, Tatsuno K, Asthana S, Jalbert L E, Nelson S J, Bollen A W, Gustafson W C, Charron E, et al. Mutational Analysis Reveals the Origin and Therapy-Driven Evolution of Recurrent Glioma. Science. 2013.

10. Phillips H S, Kharbanda S, Chen R, Forrest W F, Soriano R H, Wu T D, Misra A, Nigro J M, Colman H, Soroceanu L, Williams P M, Modrusan Z, Feuerstein B G and Aldape K. Molecular subclasses of high-grade glioma predict prognosis, delineate a pattern of disease progression, and resemble stages in neurogenesis. Cancer cell. 2006; 9(3):157-173.

11. Verhaak R G, Hoadley K A, Purdom E, Wang V, Qi Y, Wilkerson M D, Miller C R, Ding L, Golub T, Mesirov J P, Alexe G, Lawrence M, O'Kelly M, Tamayo P, Weir B A, Gabriel S, et al. Integrated genomic analysis identifies clinically relevant subtypes of glioblastoma characterized by abnormalities in PDGFRA, IDH1, EGFR, and NFL Cancer cell. 2010; 17(1):98-110.

12. Yan H, Parsons D W, Jin G, McLendon R, Rasheed B A, Yuan W, Kos I, Batinic-Haberle I, Jones S, Riggins G J, Friedman H, Friedman A, Reardon D, Herndon J, Kinzler K W, Velculescu V E, et al. IDH1 and IDH2 mutations in gliomas. The New England journal of medicine. 2009; 360(8):765-773.

13. Griewank K G, Murali R, Schilling B, Scholz S, Sucker A, Song M, Susskind D, Grabellus F, Zimmer L, Hillen U, Steuhl K P, Schadendorf D, Westekemper H and Zeschnigk M. TERT promoter mutations in ocular melanoma distinguish between conjunctival and uveal tumours. British journal of cancer. 2013; 109(2):497-501.

14. Horn S, Figl A, Rachakonda P S, Fischer C, Sucker A, Gast A, Kadel S, Moll I, Nagore E, Hemminki K, Schadendorf D and Kumar R. TERT promoter mutations in familial and sporadic melanoma. Science. 2013; 339(6122):959-961.

15. Huang F W, Hodis E, Xu M J, Kryukov G V, Chin L and Garraway L A. Highly recurrent TERT promoter mutations in human melanoma. Science. 2013; 339(6122):957-959.

16. Killela P J, Reitman Z J, Jiao Y, Bettegowda C, Agrawal N, Diaz L A, Jr., Friedman A H, Friedman H, Gallia G L, Giovanella B C, Grollman A P, He T C, He Y, Hruban R H, Jallo G I, Mandahl N, et al. TERT promoter mutations occur frequently in gliomas and a subset of tumors derived from cells with low rates of self-renewal. Proceedings of the National Academy of Sciences of the United States of America. 2013; 110(15):6021-6026.

17. Koelsche C, Sahm F, Capper D, Reuss D, Sturm D, Jones D T, Kool M, Northcott P A, Wiestler B, Bohmer K, Meyer J, Mawrin C, Hartmann C, Mittelbronn M, Platten M, Brokinkel B, et al. Distribution of TERT promoter mutations in pediatric and adult tumors of the nervous system. Acta neuropathologica. 2013.

18. Liu X, Wu G, Shan Y, Hartmann C, von Deimling A and Xing M. Highly prevalent TERT promoter mutations in bladder cancer and glioblastoma. Cell cycle. 2013; 12(10):1637-1638.

19. Vinagre J, Almeida A, Populo H, Batista R, Lyra J, Pinto V, Coelho R, Celestino R, Prazeres H, Lima L, Melo M, da Rocha A G, Preto A, Castro P, Castro L, Pardal F, et al. Frequency of TERT promoter mutations in human cancers. Nature communications. 2013; 4:2185.

20. Kim N W, Piatyszek M A, Prowse K R, Harley C B, West M D, Ho P L, Coviello G M, Wright W E, Weinrich S L and Shay J W. Specific association of human telomerase activity with immortal cells and cancer. Science. 1994; 266(5193):2011-2015.

21. Shay J W and Bacchetti S. A survey of telomerase activity in human cancer. European journal of cancer. 1997; 33(5):787-791.

22. Arita H, Narita Y, Fukushima S, Tateishi K, Matsushita Y, Yoshida A, Miyakita Y, Ohno M, Collins V P, Kawahara N, Shibui S and Ichimura K. Upregulating mutations in the TERT promoter commonly occur in adult malignant gliomas and are strongly associated with total 1p19q loss. Acta neuropathologica. 2013; 126(2):267-276.

23. Nonoguchi N, Ohta T, Oh J E, Kim Y H, Kleihues P and Ohgaki H. TERT promoter mutations in primary and secondary glioblastomas. Acta neuropathologica. 2013.

24. Marian C O, Cho SK, McEllin B M, Maher E A, Hatanpaa K J, Madden C J, Mickey B E, Wright W E, Shay J W and Bachoo R M. The telomerase antagonist, imetelstat, efficiently targets glioblastoma tumor-initiating cells leading to decreased proliferation and tumor growth. Clinical cancer research : an official journal of the American Association for Cancer Research. 2010; 16(1):154-163.

25. Ruden M and Puri N. Novel anticancer therapeutics targeting telomerase. Cancer treatment reviews. 2013; 39(5):444-456.

26. Xia W, Wang P, Lin C, Li Z, Gao X, Wang G and Zhao X. Bioreducible polyethylenimine-delivered siRNA targeting human telomerase reverse transcriptase inhibits HepG2 cell growth in vitro and in vivo. Journal of controlled release: official journal of the Controlled Release Society. 2012; 157(3):427-436.

27. Zhang P H, Zou L and Tu Z G. RNAi-hTERT inhibition hepatocellular carcinoma cell proliferation via decreasing telomerase activity. J Surg Res. 2006; 131(1):143-149.

28. Ohgaki H and Kleihues P. Genetic alterations and signaling pathways in the evolution of gliomas. Cancer science. 2009; 100(12):2235-2241.

29. Bigner S H, Matthews M R, Rasheed B K, Wiltshire R N, Friedman H S, Friedman A H, Stenzel T T, Dawes D M, McLendon R E and Bigner D D. Molecular genetic aspects of oligodendrogliomas including analysis by comparative genomic hybridization. The American journal of pathology. 1999; 155(2):375-386.

30. Cairncross G and Jenkins R. Gliomas with 1p/19q codeletion: a.k.a. oligodendroglioma. Cancer journal. 2008; 14(6):352-357.

31. Jenkins R B, Blair H, Ballman K V, Giannini C, Arusell R M, Law M, Flynn H, Passe S, Felten S, Brown P D, Shaw E G and Buckner J C. A t(1;19)(q10;p10) mediates the combined deletions of 1p and 19q and predicts a better prognosis of patients with oligodendroglioma. Cancer research. 2006; 66(20):9852-9861.

32. Smith J S, Perry A, Borell T J, Lee HK, O'Fallon J, Hosek SM, Kimmel D, Yates A, Burger P C, Scheithauer B W and Jenkins R B. Alterations of chromosome arms 1p and 19q as predictors of survival in oligodendrogliomas, astrocytomas, and mixed oligoastrocytomas. Journal of clinical oncology: official journal of the American Society of Clinical Oncology. 2000; 18(3):636-645.

33. Sjoblom T, Jones S, Wood L D, Parsons D W, Lin J, Barber T D, Mandelker D, Leary R J, Ptak J, Silliman N, Szabo S, Buckhaults P, Farrell C, Meeh P, Markowitz S D, Willis J, et al. The consensus coding sequences of human breast and colorectal cancers. Science. 2006; 314(5797):268-274.

34. Reifenberger J, Reifenberger G, Liu L, James C D, Wechsler W and Collins V P. Molecular genetic analysis of oligodendroglial tumors shows preferential allelic deletions on 19q and 1p. The American journal of pathology. 1994; 145(5):1175-1190.

1. A method of characterizing brain tumors of patients, comprising:
testing TERT promoter, IDH1, and IDH2 of a sample from a brain tumor patient, and determining mutation status (a) at mutational hot spots C228 and C250 of the TERT promoter, (b) at residue R132 of IDH1 or the codon encoding said residue, and (c) at residue R172 of IDH2 or the codon encoding said residue;
assigning a patient sample to a group that has the same mutational status at (a), (b), and (c) as the patient sample.

2. The method of claim 1 wherein the mutation status is TERT promoter wild-type and IDH1/IDH2 wild-type.

3. The method of claim 1 wherein the mutation status is TERT promoter wild-type and IDH1 or IDH2 mutant.

4. The method of claim 1 wherein the mutation status is TERT promoter mutant and IDH1 and IDH2 wild-type.

5. The method of claim 1 wherein the mutation status is TERT promoter mutant and IDH1 or IDH2 mutant.

6. The method of claim 1 further comprising assigning a prognosis to the patient.

7. The method of claim 1 wherein DNA in the sample is tested.

8. The method of claim 7 wherein DNA in the sample is amplified.

9. The method of claim 7 wherein exon 4 of IDH1 or IDH2 is tested.

10. The method of claim 8 wherein exon 4 of IDH1 or IDH2 is amplified.

11. The method of claim 1 wherein the brain tumor is a glioma.

12. The method of claim 1 wherein the brain tumor is selected from the group consisting of astrocytoma, oligodendroglioma, and glioblastoma.

13. The method of claim 1 wherein the brain tumor is an oligoastrocytoma.

14. The method of claim 1 wherein protein in the sample is tested.

15. A device for characterizing brain tumors, comprising:
one or more solid supports comprising hybridization probes specific for IDH1 codon 132, IDH2 codon 172, and TERT promoter mutation hot spots C228 and C250.

16. The device of claim 15 which consists of hybridization probes specific for less than 25 genes.

17. The device of claim 15 wherein the hybridization probes are mutation-specific.

18. The device of claim 15 wherein the hybridization probes are complementary to nucleotides adjacent to the hot spots.

19. A kit for detecting IDH1, IDH2, and TERT promoter mutations, comprising:
nucleic acid primers and/or nucleic acid probes which specifically amplify or hybridize to IDH1, IDH2, and TERT promoter mutation hot spots IDH1 R132, IDH2 R172, TERT promoter C228, and TERT promoter C250.

20. The kit of claim 19 which comprises mutation specific probes.

21. The kit of claim 19 which comprises mutation specific primers.

22. The kit of claim 19 which comprises nucleic acid primers and nucleic acid probes.

23. The kit of claim 19 which comprises deoxyribonucleotides.

24. The kit of claim 19 which comprises dideoxyribonucleotides.

25. The kit of claim 19 which comprises a DNA polymerase.

26. The kit of claim 19 which comprises nucleic acid primers, deoxyribonucleotides, dideoxyribonucleotides, and a DNA polymerase.

27. A method of characterizing brain tumors of patients, consisting of:
testing TERT promoter, IDH1, and IDH2 of a sample from a brain tumor patient, and determining mutation status (a) at mutational hot spots C228 and C250 of the TERT promoter, (b) at residue R132 of IDH1 or the codon encoding said residue, and (c) at residue R172 of IDH2 or the codon encoding said residue.

28. The device of claim 15 wherein the hybridization probes on said one or more solid supports consist of hybridization probes specific for IDH1 codon 132, IDH2 codon 172, and TERT promoter mutation hot spots C228 and C250.

29. The kit of claim 19 wherein the nucleic acid primers and/or nucleic acid probes consist of nucleic acid primers and/or nucleic acid probes which specifically amplify or hybridize to IDH1, IDH2, and TERT promoter mutation hot spots IDH1 R132, IDH2 R172, TERT promoter C228, and TERT promoter C250.

30. The method of claim 1 wherein the sample is tested for a mutation at sites consisting of (a), (b), and (c).
